(19) United States
(12) Patent Application Publication
(10) Pub. No.: US 2008/0127360 A1
(43) Pub. Date: May 29, 2008

(54) Swine Cell For Xenotransplantation, Method of Selecting the Same and Swine For Xenotransplantation

(75) Inventors: Yoichi Takahagi, Ibaraki (JP); Tatsuya Fujimura, Ibaraki (JP); Hiroshi Murakami, Ibaraki (JP); Tamotsu Shigehisa, Ibaraki (JP)

Correspondence Address: BIRCH STEWART KOLASCH & BIRCH, PO BOX 747, FALLS CHURCH, VA, 22040-0747, US

(73) Assignee: THE ANIMAL ENGINEERING RESEARCH INSTITUTE, Ibaraki (JP)

(21) Appl. No.: 11/663,356

(22) PCT Filed: Mar. 25, 2005
(86) PCT No.: PCT/JP05/06452

§ 371 Date: Oct. 25, 2007

(30) Foreign Application Priority Data
Nov. 2, 2004 (JP) 2004-319939

(51) Int. Cl. A01K 67/00; C12N 5/10; G01N 33/567

(52) U.S. Cl. 800/8; 435/325; 435/7.21

(57) ABSTRACT

The invention presents a swine cell not expressing α-1,3-galactosyl transferase and expressing human complement inhibitor and/or human N-acetyl glucosaminyl transferase III; a method of selecting the same; and a swine for xenotransplantation. The swine for xenotransplantation of the invention is very useful in inhibiting the acute rejection characteristic to xenotransplantation because it does not express α-Gal antigen but inhibits the progress of a complement reaction, if unexpectedly induced, and/or expresses little xenoantigen other than α-Gal antigen.

### TECHNICAL FIELD

The present invention relates to a swine cell for xenotransplantation, method of selecting the same, and swine for xenotransplantation. More particularly, the invention relates to a swine cell not expressing α-1,3-galactosyl transferase (hereinafter referred to GT), but expressing human complement inhibitor and/or human N-acetyl glucosamine transferase III (β-1,4-mannosyl-glycoprotein β-1,4-N-acetyl glucosaminyl transferase, EC2.4.1.144, hereinafter referred to GnT-III), its selecting method, and swine for xenotransplantation not causing acute rejection characteristic to xenotransplantation.

### BACKGROUND ART

In treatment of patients having dysfunction in organ, tissue or cell, organ transplantation is a very useful therapy, and already kidney transplantation, liver transplantation and heart plantation are operated widely. Organ transplantation is classified into allotransplantation and xenotransplantation (grafting, embedding, injecting or contacting of living organ, tissue or cell of a donor of different species from the recipient species into the recipient species), and each transplantation has its own merits and demerits. Allotransplantation is lower in possibility of rejection as compared with xenotransplantation, but if the recipient is human, the number of donors is limited, and hence xenotransplantation is researched and developed more intensively. As the donor animal for xenotransplantation, a swine is considered to be most preferable because the organ size and morphology of swine are close to those of the human and the breeding and rearing technology is established.

When swine organs (for example, heart, lung, liver, kidney, pancreas, skin), tissues (for example, coronary artery, brain dura mater, cartilage, cornea), or cells (for example, islet of Langerhans of pancreas, nigrostriatal cell of brain) (hereinafter referred to graft) are transplanted in the human subject, the grafts are rejected in a short period. Such acute rejection characteristic to xenotransplantation is called hyperacute rejection or acute vascular rejection, and it is caused by reaction between xenoantigen existing in the swine graft and natural antibody in human serum, and subsequent reactions triggered by this reaction such as complement dependent cell damaging reaction, activation of vascular endothelium, clotting reaction, NK cell dependent cell damaging reaction, etc.

Principal xenoantigen in the swine graft is galactose α-1,3-galactose (hereinafter referred to α-Gal antigen) existing in non-reducing terminal of swine cell sugar chain (sugar protein, sugar lipid), and by the action of GT, α-Gal antigen is synthesized by α-1,3 bonding of galactose on N-acetyl lactosamine. In the case of Old World apes (Cercopithesidae), anthropoids and humans, GT genes are transformed into pseudogenes (see Non-patent document 1), and α-Gal antigen is not produced, and to the contrary, the antibody to α-Gal antigen (hereinafter referred to anti-α-Gal antibody) is produced and present.

Non-patent document 1: J. Biol. Chem., 1990: 265; 7055-61

To avoid the acute rejection characteristic to xenotransplantation, treatment on transplantation recipient (human) and treatment on donor animal (swine) are known. The former includes a method of adsorbing and removing natural antibodies in the recipient blood, and a method of administering anti-α-Gal antibody neutralizing agent. The latter includes a method of expressing complement inhibitor of recipient species in the donor animal (see, for example, Patent document 1, Patent document 2), and a method of decreasing or not expressing α-Gal antigen of donor animal.

Patent document 1: Japanese unexamined patent publication No. H05-503074

Patent document 2: Japanese unexamined patent publication No. H11-239430

The method of decreasing or not expressing α-Gal antigen of donor animal includes the following.
(1) Method of decreasing α-Gal antigen by acting α-galactosidase for dissecting α-galactoside bond of α-Gal antigen;
(2) Method of decreasing α-Gal antigen by acting or expressing endoβ-galactosidase for dissecting β-galactoside bond of α-Gal antigen (see Patent document 3);

Patent document 3: Japanese unexamined patent publication No. 01-029200
(3) Method of competitive inhibition of expression of α-Gal antigen by forced expression of the sugar transferase (for example, α-1,2-fucosyl transferase) other than GT acting on substrate (N-acetyl lactosamine) in common with GT (see Patent document 4);

Patent document 4: WO 95/34202 A1
(4) Method of inhibiting for synthesis of α-Gal antigen and N-linked sugar chain other than α-Gal antigen by expressing GnT-III capable of preventing N-linked sugar chain from branching (see Patent document 5); and

Patent document 5: Japanese unexamined patent publication No. 2002-291372
(5) Method of knocking out (KO) GT gene so as not to express α-Gal antigen (see, for example, Patent documents 6 to 12).

Patent document 6: Japanese unexamined patent publication No. H8-509603

Patent document 7: Japanese unexamined patent publication No. H9-508277

Patent document 8: Japanese unexamined patent publication No. H10-504442

Patent document 9: Japanese unexamined patent publication No. 2002-17360

Patent document 10: WO 01/88096

Patent document 11: WO 03/055302

Patent document 12: WO 03/064658

Preparation of gene knock-out mammalian animal was hitherto possible only in the mouse in which the embryonic stem cell line was established, but ever since the development of cloned sheep preparation technology (see Non-patent document 2) by somatic nuclear transfer, and more specifically, ever since the development of gene knock-out sheep preparation technology (see Non-patent document 3) by somatic nuclear transfer, it is possible also in other mammalian animals than the mouse.

Non-patent document 2: Nature 1996: 380; 64-6

Non-patent document 3: Nature Biotechnol. 2001: 19; 559-62

From the transplantation test in primates, it has been found that the swine graft prepared in the methods of (1) to (4) cannot overcome the acute rejection characteristic to xenotransplantation. The duration up to rejection of the swine graft prepared in the method of (5) in baboon was not significantly different from that of the transgenic swine graft expressing the human complement inhibitor developed hitherto (see Non-patent document 4).

Non-patent document 4: Organ Biol. 2004: 11; 11-19

Possible factors are estimated as follows: the swine cell includes a second α-1,3 GT responsible for synthesis of α-Gal antigen (see Non-patent document 5), α-Gal antigen is remaining even in the swine cell knocking out the GT gene (about 1-2% of normal level, see Non-patent document 6), and other xenoantigen than α-Gal antigen is also expressed in the swine cell (for example, H-D antigen, see Non-patent document 7).

Non-patent document 5: Xenotransplantation 2001: 8 (Supplement 1); 27

Non-patent document 6: Transplantation 2003: 75; 430-6

Non-patent document 7: Xenotransplantation 2004: 11; 237-46

Hence, there has been a keen demand for swine cell and swine for xenotransplantation, not expressing α-Gal antigen, capable of suppressing the progress if complement reaction is induced and/or decreasing xenoantigen other than α-Gal antigen, and capable of suppressing the acute rejection characteristic to xenotransplantation.

In order not to express α-Gal antigen in the swine graft, as shown in Examples below, it is not enough by knocking out the GT gene of one allele only (hereinafter referred to single GTKO), but it is required to knock out the GT genes of both alleles (hereinafter referred to double GTKO). Hence, double GTKO swine was prepared (see, for example, Non-patent documents 8 and 9).

Non-patent document 8: Science 2003: 299; 411-414

Non-patent document 9: PNAS 2004: 101; 7335-40

The double GTKO swine was prepared in the following procedure: a targeting vector for GTKO was introduced in the swine somatic cell sampled from ordinary nontransgenic swine, and a homologous recombinant single GTKO swine cell was prepared and selected, and using the single GTKO swine cell, a single GTKO clone swine embryo was prepared, and the single GTKO clone swine embryo was transplanted in recipient gilt, and a fetus of single GTKO swine was prepared, and the cell sampled from the single GTKO swine fetus was transformed and a double GTKO swine cell was prepared and selected, and using the double GTKO swine cell, a double GTKO cloned swine embryo was prepared, and the double GTKO cloned swine embryo was transplanted in recipient gilt.

In preparation of the double GTKO swine, the method of selecting the double GTKO swine cell from a mixture of the single GTKO swine cell (expressing α-Gal antigen) and the double GTKO swine cell (not expressing α-Gal antigen) includes the following:
(1) A method of destroying the single GTKO swine cell by acting anti-α-Gal antibody and complement (hereinafter referred to complement selection method; see Non-patent documents 6 and 9 mentioned before), and
(2) A method of destroying the single GTKO swine cell by acting Toxin A of Clostridium difficile having affinity for α-Gal antigen and cytotoxicity (hereinafter referred to Toxin A method; see Non-patent document 8 mentioned before).

If attempted to prepare the double GTKO swine cell by using somatic cell of transgenic swine expressing human complement inhibitor, the cell suppresses the complement dependent cytotoxicity by the function of the generated human complement inhibitor, and the complement selection method cannot be applied directly when selecting the single GTKO swine cell and double GTKO swine cell expressing human complement inhibitor.

In the transgenic swine expressing GnT-III, since the expression amount of α-Gal antigen is decreased by the function of expressed GnT-III, it is not efficient if Toxin A method is applied in selection of the single GTKO swine cell and double GTKO swine cell.

Hence, the double GTKO swine cell expressing human complement inhibitor and/or human GnT-III and its efficient selecting method have been considered necessary. At the same time, there has been a keen demand for swine for xenotransplantation prepared by presenting such swine cell to somatic cell nuclear transfer, and not expressing α-Gal antigen, capable of suppressing the progress if complement reaction is induced and/or decreasing xenoantigen other than α-Gal antigen, and capable of suppressing the acute rejection characteristic to xenotransplantation.

### DISCLOSURE OF THE INVENTION

The invention is hence devised to solve the problems of the prior arts, and is intended to present a swine cell not expressing GT and expressing human complement inhibitor and/or human GnT-III, its selecting method, and swine for xenotransplantation not causing the acute rejection characteristic to xenotransplantation.

To solve the problems, the invention presents:
(1) A swine cell not expressing GT and expressing human complement inhibitor and/or human GnT-III;
(2) A selecting method of a double GTKO swine cell expressing human complement inhibitor and/or human GnT-III characterized by removing a single GTKO swine cell expressing human complement inhibitor and/or human GnT-III and obtaining the double GTKO swine cell expressing human complement inhibitor and/or human GnT-III by causing
(a) a substance specifically bonding with α-Gal antigen bonded with a carrier; or
(b) one substance of a pair of substances having bonding property bonded with a substance specifically bonding with α-Gal antigen, and other substance of the pair of substances having bonding property bonded with a carrier; to act on
   a mixture of the single GTKO swine cell expressing human complement inhibitor and/or human GnT-III and the double GTKO swine cell expressing human complement inhibitor and/or human GnT-III;
(3) A selecting method of the double GTKO swine cell expressing human complement inhibitor and/or human GnT-III characterized by removing a single GTKO swine cell expressing human complement inhibitor and/or human GnT-III and obtaining a double GTKO swine cell expressing human complement inhibitor and/or human GnT-III by acting anti-human complement inhibitor antibody on a mixture of the single GTKO swine cell expressing human complement inhibitor and/or human GnT-III and the double GTKO swine cell expressing the same, and then acting anti-α-Gal antibody and complement on the resultant mixture; and

(4) A swine for xenotransplantation having a swine cell not expressing GT and expressing human complement inhibitor and/or human GnT-III.

### BRIEF DESCRIPTION OF THE DRAWINGS

FIG. 1 shows a targeting vector for knocking out swine α-1,3-galactosyl transferase gene. The figure is an example using exon 9 as the swine GT gene.

FIG. 2 shows wild type, single knock-out type and double knock-out type of swine α-1,3-galactosyl transferase gene by Southern blotting method.

FIG. 3 shows the result of α-Gal antigen amount by FACS analysis.

### BEST MODE FOR CARRYING OUT THE INVENTION

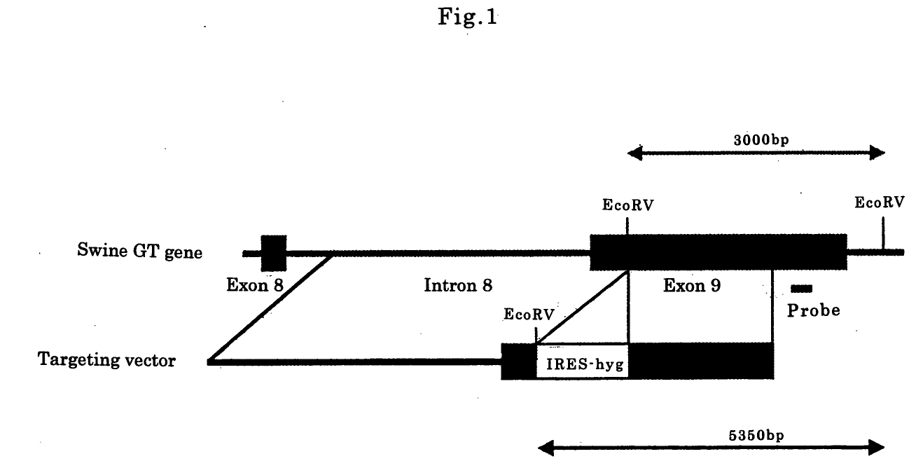

The swine cell and swine for xenotransplantation of the invention not expressing α-Gal antigen, capable of suppressing the progress if complement reaction is induced and/or decreasing xenoantigen other than α-Gal antigen, and capable of suppressing the acute rejection characteristic to xenotransplantation are the swine cell and swine for xenotransplantation not expressing GT, and expressing human complement inhibitor and/or human GnT-III, and which can be prepared as follows.
(1) In cells sampled from transgenic swine (Patent document 2 mentioned before) expressing a transgene comprising a regulatory gene expressed in swine organ and tissue, especially vascular endothelial cell and human complement inhibitor (DAF/CD55) gene, or transgenic swine (Patent document 5 mentioned before) expressing a transgene comprising a regulatory gene functioning in the hyperacute rejection-occurring local cells, human GnT-III gene and human complement inhibitor gene, a GTKO targeting vector (see FIG. 1) described below is introduced, and homologous recombinant single GTKO swine cells are prepared and selected, and
   single GTKO cloned swine embryos are prepared by nuclear transfer using the single GTKO swine cells as donor cells and denucleated unfertilized swine ova as recipient ova, and
   the single GTKO cloned swine embryos are transplanted in recipient gilts, cells are sampled from fetuses or piglets of the single GTKO swine, and cultivated, and
   the cultured cells are transformed, and double GTKO swine cells are prepared and selected, and double GTKO cloned swine embryos are prepared by using the double GTKO swine cells as donor cells, and
   the double GTKO cloned swine embryos are transplanted in recipient gilts, and piglets not expressing GT and expressing human complement inhibitor and/or human GnT-III are prepared;
(2) (a) In cells sampled from the previously prepared double GTKO swine, a transgene comprising a regulatory gene expressed in swine organ and tissue, especially vascular endothelial cell, and human complement inhibitor gene, or a transgene comprising a regulatory gene functioning in the hyperacute rejection-occurring local cells, human GnT-III gene and human complement inhibitor gene is introduced, and the double GTKO swine cells not expressing GT and expressing human complement inhibitor and/or human GnT-III are prepared and selected, and the double GTKO cloned swine embryos are prepare by using the double GTKO swine cells as donor cells, and the double GTKO cloned swine embryos are transplanted in recipient gilts; or

(b) the previously prepared double GTKO swine is mated with the previously prepared transgenic swine expressing human complement inhibitor especially in vascular endothelial cell or the previously prepared transgenic swine expressing human GnT-III; and thereby

the piglets not expressing GT and expressing human complement inhibitor and/or human GnT-III are prepared; and
(3) (a) the single GTKO swine (male and female) of (1) are mated upon sexual maturity; or

(b) the single GTKO swine (male or female) of (1) is mated with ordinary swine upon sexual maturity, and F1 (male and female) of the single GTKO swine are obtained, and these F1 single GTKO swine are mated upon sexual maturity; and thereby,

the piglets not expressing GT and expressing human complement inhibitor and/or human GnT-III are prepared.

The human complement inhibitor in the swine cell not expressing GT and expressing human complement inhibitor and/or human GnT-III is not particularly specified as far as it is human complement inhibitor, and examples include DAF (CD55), MCP (CD46) and CD59.

The method of (1) mentioned above is the most preferred embodiment of the invention because time, labor and expenses required for preparation of the double GTKO swine can be saved substantially, the transgene of human complement inhibitor (DAF/CD55) and/or human GnT-III can be securely transmitted to the offspring, and the functional protein can be securely expressed. The selection method of double GTKO swine cells of the invention can be applied not only to (1) but also to (2).

Cells sampled from the transgenic swine in (1) and cells samples from the double GTKO swine in (2) are not particularly specified in type, but preferred examples are epithelial cell, fibroblast, endothelial cell, muscular cell, nervous cell, and tissue stem cell, and the fibroblast is particularly preferred.

DNA sequence (targeting vector; see FIG. 1) for inactivating swine GT gene (gene knock-out; KO) by homologous recombination with swine GT is composed of:
(a) DNA sequence homologous to one or more exons of the group selected from exon 4, exon 5, exon 6, exon 7, exon 8 and exon 9 of swine GT gene (Patent documents 6 and 7),
(b) DNA sequence homologous to intron of 5′ terminal and/or 3′ terminal of above (a), and
(c) DNA sequence not homologous to (a) in order to inhibit the function of (a).

The DNA sequence of above (c) is composed of DNA sequence of drug resistant gene and/or DNA sequence for starting and ending expression of the drug resistant gene. The drug resistant gene includes neomycin resistant gene, puromycin resistant gene, hygromycin (Hyg) resistant gene and the like. The DNA sequence for starting expression of the drug resistant gene includes IRES (internal ribosome entry site), Kozak sequence and the like. The DNA sequence for ending expression of the drug resistant gene includes poly A sequence and the like. In the targeting vector, limiting enzyme site may be properly disposed. The DNA sequence of intron of above (b) is preferred to be isogenic.

The targeting vector can be introduced into the swine cell by electroporation method, or by using chemical drug such as LipofectAMINE (trademark).

The single GTKO swine cell by homologous recombination of swine GT gene and the targeting vector can be isolated as colony by cultivating and selecting with culture medium including drug of proper concentration corresponding to the type of drug resistant gene incorporated in the targeting vector. The DNA of the isolated colony is genetically analyzed by PCR, Southern blotting or other method, and confirmed to be the single GTKO swine cell and normal karyotype, and then it can be presented for process of loss of heterozygosity or preparation of the single GTKO swine embryo as donor cell described below.

The GTKO cloned swine embryo and GTKO swine can be prepared as follows.

From the follicles of swine ovaries sampled at slaughterhouse, swine ovum-cumulus oophorus cell complexes are sucked and sampled, and cultivated in culture medium (NCSU-23 culture medium with or without cysteine, EGF, eCG, hCG and swine follicle solution), and the cumulus oophorus cells are removed by hyaluronidase treatment, the nuclei of ova are removed by sucking and removing the cytoplasm near the releasing position of first polar body, and one cell of the single GTKO swine cell or the double GTKO swine cell is inserted into one ovum (denucleated swine ovum) around the ovum cavity, and electrofused, and by activating at the same time or after electrofusion, the cloned swine embryo can be prepared. Activation may be carried out by electric treatment or chemical treatment.

Recipient gilts preliminarily treated with hormones (eCG, hCG) are laparotomized under anesthesia, and the cloned swine embryos are injected into the swollen portion of oviduct, the section is sutured, and when the transplanted cloned swine embryos can be implanted, swine fetuses or piglets are obtained after the conception period or gestation cycle. By sampling part of swine skin, by various gene analyses (for example, PCR, Southern blotting, Northern blotting, microsatellite analysis), the single GTKO swine or the double GTKO swine can be identified. All animals are handled according to the spirit of animal welfare and rules of related regulations.

By cultivating the single GTKO swine cells, the double GTKO swine cells may be produced by variation occurring at a certain probability. Such phenomenon is known as loss of heterozygosity (LOH) (Am. J. Hum. Genet. 1997: 61; 995-9).

As the single GTKO swine cell to be presented for LOH treatment, it is preferred to use the single GTKO swine cell prepared from the single GTKO swine fetus or the single GTKO piglet, because implantation ability in recipient gilt, initial generation ability, and reproductivity are confirmed.

As the method of selecting the double GTKO swine cells only from a mixture of the single GTKO swine cells (expressing α-Gal antigen) and the double GTKO swine cells (not expressing α-Gal antigen), generally, (1) a method of destroying by lysis the single GTKO swine cells by acting anti-α-Gal antibody and complement (complement selection method), and (2) a method of destroying the single GTKO swine cells by acting Toxin A of Clostridium difficile having affinity for α-Gal antigen and cytotoxicity (Toxin A method) are applied.

However, the GTKO swine cells expressing human complement inhibitor of the invention are expressing human complement inhibitor whether the single GTKO or the double GTKO, direct selection by human complement selection method cannot be applied. On the other hand, the GTKO swine cells expressing human GnT-III of the invention are decreased in the expressing amount of α-Gal antigen even in the single GTKO swine cells, and the bonding amount of Toxin A and cytotoxicity are decreased, and efficient selection is impossible by the Toxin A method.

Considering from such problems, the present inventors investigated into the method of selectively sorting the double GTKO swine cells expressing human complement inhibitor and/or human GnT-III from a mixture of the single GTKO swine cells expressing human complement inhibitor and/or human GnT-III and the double GTKO swine cells of the same, and found that the object can be achieved in the following methods. That is,
(1) A selecting method of the double GTKO swine cells expressing human complement inhibitor and/or human GnT-III characterized by removing the single GTKO swine cells expressing human complement inhibitor and/or human GnT-III and obtaining the double GTKO swine cells expressing human complement inhibitor and/or human GnT-III by causing

(a) a substance specifically bonding with α-Gal antigen bonded with a carrier; or

(b) one substance of a pair of substances having bonding property bonded with a substance specifically bonding with α-Gal antigen, and other substance of the pair of substances having bonding property bonded with a carrier; to act on

the mixture of the single GTKO swine cells expressing human complement inhibitor and/or human GnT-III and the double GTKO swine cells expressing human complement inhibitor and/or human GnT-III; or
(2) A selecting method of the double GTKO swine cells expressing human complement inhibitor and/or human GnT-III characterized by removing the single GTKO swine cells expressing human complement inhibitor and/or human GnT-III and obtaining the double GTKO swine cells expressing human complement inhibitor and/or human GnT-III by causing anti-human complement inhibitor antibody to act on the mixture of the single GTKO swine cells expressing human complement inhibitor and/or human GnT-III and the double GTKO swine cells of the same and then anti-α-Gal antibody and complement to act on the resultant mixture.

In the method of (1), the substance specifically bonding with α-Gal antigen includes, for example, α-Gal antigen specific lectin (Griffonia simplicifolia 1 isolectin B4; GS1 B4), anti-α-Gal antigen antibody, etc.

As the carrier for bonding the substance, common carrier (for example, Sepharose) used in the biochemical field may be used, and it is particularly preferred to use magnetic beads because of ease of handling.

The pair of substances having bonding property include combination of avidin (or streptoavidin) and biotin (or iminobiotin, dithiobitin, etc.), and combination of concanavalin A and sugars (for example, glucose, etc.), and the pair of substances having bonding property are known to those skilled in the art.

Bonding of the carrier and the substance specifically bonded with α-Gal antigen, bonding of the substance of the pair of substances having bonding property with the carrier, and bonding of the same substance and the substance specifically bonded with α-Gal antigen can be carried out by conventional method such as chemical boding method and the like.

Examples of selecting method of the invention are specifically described below.
(1) Magnetic Beads Method

In the mixture of the single GTKO swine cells and the double GTKO swine cells expressing human complement inhibitor and/or human GnT-III,

(a) magnetic beads labeled α-Gal antigen specific lectin (Griffonia simplicifolia 1 isolectin B4; GS1 B4) is caused to act; or

(b) biotin labeled GS1 B4 lectin and streptoavidin labeled magnetic beads are caused to act; or

(c) streptoavidin labeled GS1 B4 lectin and biotin labeled magnetic beads are caused to act; then

the single GTKO swine cells expressing α-Gal antigen bonded with the magnetic beads are removed by the action of magnet, so that the double GTKO swine cells can be selected.

Instead of the GS1 B4 lectin, α-Gal antigen antibody may be used, and specifically in (a), (b), and (c), respectively, magnetic beads labeled anti-α-Gal antigen antibody, biotin labeled anti-α-Gal antigen antibody and streptoavidin labeled magnetic beads, and streptoavidin labeled anti-α-Gal antigen antibody and biotin labeled magnetic beads may be used.

Instead of the magnetic beads, other common carriers may be also used.
(2) Combined Use Method of Anti-Human Complement Inhibitor Antibody

In the mixture of the single GTKO swine cells and the double GTKO swine cells expressing human complement inhibitor and/or human GnT-III, anti-human complement inhibitor antibody is caused to act to block human complement inhibitor, and anti-α-Gal antibody and complement (for example, human serum) are applied and the single GTKO swine cells are destroyed by lysis, so that the double GTKO swine cells can be selected.

The anti-human complement inhibitor antibody used in the combined use method of anti-human complement inhibitor antibody must be an antibody not having complement receptor, such as Fab fraction of IgG, IgY, etc. The antibody used in the combined use method of anti-human complement inhibitor antibody is not limited to the human antibody alone.

By using the swine cells not expressing GT and expressing human complement inhibitor and/or human GnT-III thus selected, when the manufacturing method of the cloned GTKO swine embryo and the GTKO swine is applied, the swine cells not expressing GT and expressing human complement inhibitor and/or human GnT-III and the swine for xenotransplantation, that is, the swine for xenotransplantation not expressing α-Gal antigen, and capable of suppressing the progress if complement reaction is induced and/or decreasing xenoantigen other than α-Gal antigen, and capable of suppressing the acute rejection characteristic to xenotransplantation can be manufactured. Therefore, the organs (for example, heart, lung, liver, kidney, pancreas, skin), tissues (for example, coronary artery, brain dura mater, cartilage, cornea, nerves) and cells (for example, cardiac muscle, islet of Langerhans of pancreas, nigrostriatal cell of brain) of the swine can be preferably used as the grafts for xenotransplantation.

### INDUSTRIAL APPLICABILITY

The swine cells of the invention are the swine cells not expressing GT (that is, not expressing α-Gal antigen) and expressing human complement inhibitor and/or human GnT-III, and when these swine cells are used as donor cells in somatic cell nuclear transfer, the swine for xenotransplantation not expressing α-Gal antigen and expressing human complement inhibitor and/or human GnT-III can be presented.

The swine for xenotransplantation of the invention is the swine for xenotransplantation having swine cells not expressing α-Gal antigen and expressing human complement inhibitor and/or human GnT-III, and the organs, tissues and cells of the swine are very useful because they do not express α-Gal antigen, and can suppress the progress if complement reaction is induced and/or can decrease xenoantigen other than α-Gal antigen, and can suppress the acute rejection characteristic to xenotransplantation

The selecting method of the invention is a selecting method of the double GTKO swine cells expressing human complement inhibitor and/or human GnT-III, from the mixture of the single GTKO swine cells expressing human complement inhibitor and/or human GnT-III and the double GTKO swine cells expressing human complement inhibitor and/or human GnT-III, and according to this method, the double GTKO swine cells can be obtained certainly and easily.

### EXAMPLES

Examples of the invention are more specifically described below, but it must be noted that the invention is not limited to these Examples alone.

### Example 1

### Swine Cells Expressing Human Complement Inhibitor and/or Human N-Acetyl Glucosaminyl Transferase III (GnT-III)

Transgenic swine (Transplant. Proc. 2003: 35; 516-7) expressing human complement inhibitor and human GnT-III in local cells causing hyperacute rejection, and ordinary swine were mated, and fetus was collected on day 28 to 33 of gestation, and sectioned, treated in 0.25% trypsin-0.02% EDTA (Gibco BRL) for 30 minutes at 37° C., and cultivated in 10% bovine fetal serum added DMEM culture medium (Gibco BRL) at 37° C. and 5% CO₂, and cells having DNA of human DAF and human GnT-III and having a normal karyotype were selected.

Similarly, a transgenic swine (Mol. Reprod. Del. 2002:61;302-11) expressing human DAF at local cell where hyperacute rejection occurs is mated with an ordinary swine, and cells having human DAF and having a normal karyotype were selected.

The cells were confirmed to have DNA of human DAF or human GnT-III by the method described below, and to have a normal karyotype by the stain sample preparation method (“Technology of cell cultivation,” ed. by Japan Cell Cultivation Society).

### Example 2

### Single GTKO Swine Cell Expressing Human Complement Inhibitor and/or Human GnT-III

In the above swine cells (2×10⁶ cells), the targeting vector for GTKO (10 μg, FIG. 1) was injected by electroporation (300 V, 975 μFD), and cultivated for 1 to 4 days. Further cultivating for 7 to 14 days in culture medium with hygromycin (50 to 200 μg/ml), colonies were picked up in 20 to 30 days after introduction of the targeting vector, and single GTKO swine cells expressing human DAF and human GnT-III were selected. Similarly, single GTKO swine cells expressing human DAF were selected.

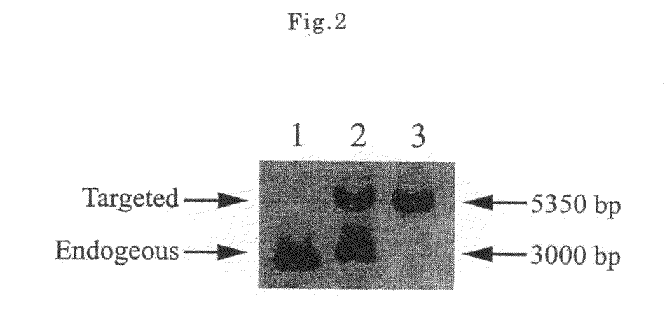

The swine cells were confirmed to be single GTKO swine cells by the Southern blotting method described below. That is, in the cells of ordinary swine (wild type), only band of 3000 bp was observed, but in the single GTKO swine cells, bands of 3000 bp and 5350 bp were observed (lanes 1 and 2 in FIG. 2). The single GTKO cells were presented for preparation of single GTKO cloned swine embryos.

### Example 3

Single GTKO Cloned Swine Embryo Expressing Human Complement Inhibitor and/or Human GnT-III

From the follicles of swine ovaries sampled at slaughterhouse, swine ovum-cumulus oophorus cell complexes (3 to 6 mm in diameter) were sucked and sampled, and cultivated in culture medium NCSU-23 with 0.6 mM cysteine, 10 ng/ml EGF, 10 IU/ml eCG, 10 IU/ml hCG and swine follicle solution (10% (v/v)) at 38.5° C. and in 5% CO₂ for 15 to 26 hours, and further cultivated in culture medium without hormones for 15 to 26 hours. By treating in hyaluronidase (1 mg/ml), the cumulus oophorus cells were removed, and the ova releasing the first polar body were sampled. By 10 mM Hepes buffer Tyrode's lactose culture medium containing 0.3% (w/v) polyvinyl pyrrolidone, 10% (v/v) bovine fetal serum and 7.5 μg/ml cytochalasin B, the cumulus oophorus cells were removed from the swine ova, and immediately by using a pipette (diameter 35 μm), the first polar body and its peripheral cytoplasm (about 10% of entire cytoplasm) were sucked, and the nuclei were removed from the swine ova (denucleated). The swine ova were confirmed to be denucleated by staining with Hoechst 33342 (5 μg/ml).

The denucleated swine ova were slowly put on Hepes buffer Tyrode's lactose culture medium containing 0.3% polyvinyl pyrrolidone, by using a pipette (diameter 20 μm), the donor cells (single GTKO swine cells expressing human DAF and human GnT-III, or single GTKO swine cells expressing human DAF) were injected in the ovum cavities, and the donor cell-ovum complexes were prepared, and placed between two electrodes, and electrically fused and activated by using an electric cell fusion device (Shimadzu). That is, in 0.27M mannitol solution containing 0.1 mM MgSO₄ and 0.05 mM CaCl₂, the donor cell-ovum complexes were treated in alternating current at 1 MHz, 5 V for 5 seconds, and in direct current at 160 V/mm for 30 μs, and fused, and cultivated in NCSU-23 culture medium containing 5 μg/ml cytochalasin B for 1 to 1.5 hours, and activated in direct current at 100 V/min for 100 μs. Or, in 0.27M mannitol solution containing 0.1 mM MgSO₄ only, they were treated in alternating current at 1 MHz, 5 V for 5 seconds, and in direct current at 180 V/mm for 10 μs, and fused, and after 1 to 1.5 hours, they were activated in direct current at 150 V/mm for 100 μs, and cultivated in NCSU-23 culture medium containing 5 μg/ml cytochalasin B for 3 hours, and single GTKO cloned swine embryos expressing human DAF and human GnT-III or single GTKO cloned swine embryos expressing human DAF were prepared.

### Example 4

### Transplantation of Single GTKO Cloned Swine Embryo Expressing Human Complement Inhibitor and/or Human GnT-III in Recipient Gilt

In trihybrid gilts (LW/L×D), 1,000 IU of eCG was intramuscularly injected, and 65 to 80 hours later, 1,500 IU of hCG was intramuscularly injected. Two or three days later, the gilts were laparotomized under anesthesia, and the cloned swine embryos were injected into the swollen portion of the oviduct, and the section was sutured. In 28 to 35 days after transplantation of the single GTKO cloned swine embryos in recipient gilts, the recipient gilts were laparotomized, and single GTKO swine fetuses expressing human DAF and human GnT-III were collected.

Similarly, single GTKO swine fetuses expressing human DAF were collected.

On day 115 after transplantation of the single GTKO cloned swine embryos expressing human DAF and human GnT-III, healthy piglets of single GTKO swine expressing human DAF and human GnT-III were obtained.

All animals were handled according to the spirit of animal welfare and rules of related regulations. The swine fetuses and piglets were confirmed to have DNA of human DAF, human GnT-III or the targeting vector by the following methods, and to have normal karyotype by the method explained above.

### Example 5

### Double GTKO Cloned Swine Cell Expressing Human Complement Inhibitor and/or Human GnT-III

The single GTKO cloned swine fetuses obtained above and expressing human DAF and human GnT-III were sectioned, and cultivated similarly, and single GTKO swine cells having normal karyotype and expressing human DAF and human GnT-III were prepared. Similarly, single GTKO swine cells having normal karyotype and expressing human DAF were prepared.
(a) Magnetic Beads Method

In the single GTKO swine cells (3-5×10⁶) expressing DAF and GnT-III obtained above, or the single GTKO swine cells (3-5×10⁶) expressing DAF obtained above, biotin labeled α-Gal antigen specific lectin (Griffonia simplicifolia 1 isolectin B4; GS1 B4; 10 μg) was caused to act (4° C., 0.5 to 2 hours), and successively, streptoavidin labeled magnetic beads were caused to act (4° C., 0.5 to 2 hours), and a magnet was applied, and excessive magnetic beads attracted to the magnet and cells bonding to the magnetic beads were removed. Cells collected in the reaction solution after removal were cultivated, and in colonies formed in 15 to 21 days, double GTKO swine cells were identified in the method mentioned below. As a result, the double GTKO swine cells expressing human DAF and human GnT-III showing only band of 5350 bp were obtained (lane 3 in FIG. 2). The rate of number of the double GTKO swine cell colonies in the number of sample colonies was 6/55 (10.9%).

Similarly, a double GTKO swine cells expressing human DAF were obtained.

Instead of biotin labeled α-Gal antigen specific lectin and streptoavidin labeled magnetic beads, when magnetic beads labeled anti-α-Gal antigen antibody was caused to act, similarly, the double GTKO swine cells could be collected.
(b) Toxin A Method

The single GTKO swine cells (5×10⁵) expressing DAF and GnT-III obtained above were cultivated in culture medium containing Toxin A (0.5 to 2 μg/ml) for 2 hours, and cultivated in ordinary culture medium, and in colonies formed in 21 to 25 days, double GTKO swine cells were identified similarly. As a result, the double GTKO swine cells expressing human DAF and human GnT-III showing only band of 5350 bp were obtained. The rate of number of the double GTKO swine cell colonies in the number of sample colonies was 2/76 (2.6%). If Toxin A method was applied in selection of the double GTKO swine cells, the same efficiency as in the magnetic beads method was not obtained (2.6% vs. 10.9%).
(c) Combined Use Method of Anti-Human Complement Inhibitor Antibody

In the single GTKO swine cells (5×10⁵) expressing human antibody complement inhibitor and human GnT-III obtained above, anti-DAF antibody (Fab fraction, 5 μg) was caused to act (4° C., 0.5 to 2 hours), and DAF was masked. After washing, they were reacted with human serum 1 ml (37° C., 0.5 to 2 hours). After reaction, the cells were recovered and cultivated. In colonies formed in 15 to 25 days, similarly, the double GTKO swine cells were identified. The efficiency of selection of the double GTKO swine cells was 8.9%.

### Example 6

### Double GTKO Swine Expressing Human Complement Inhibitor and/or Human GnT-III

According to the above method, the double GTKO cloned swine embryos were prepared from the double GTKO swine cells expressing human DAF and human GnT-III, and the double GTKO cloned embryos were transplanted in recipient gilts, and healthy piglets were obtained on day 110 after transplantation. In these piglets, presence or absence of DNA of human DAF, DNA of human GnT-III, and DNA of GTKO targeting vector was investigated together with expression of human DAF, human GnT-III and α-Gal antigen. Summing up the results, the piglets were confirmed to be the double GTKO swine expressing human DAF and human GnT-III.

Similarly, double GTKO cloned swine embryos were prepared from the double GTKO swine cells expressing human DAF, and the embryos were transplanted in recipient gilts, and healthy piglets were obtained on day 110 after transplantation, and these piglets were confirmed to be the double GTKO swine expressing human DAF.

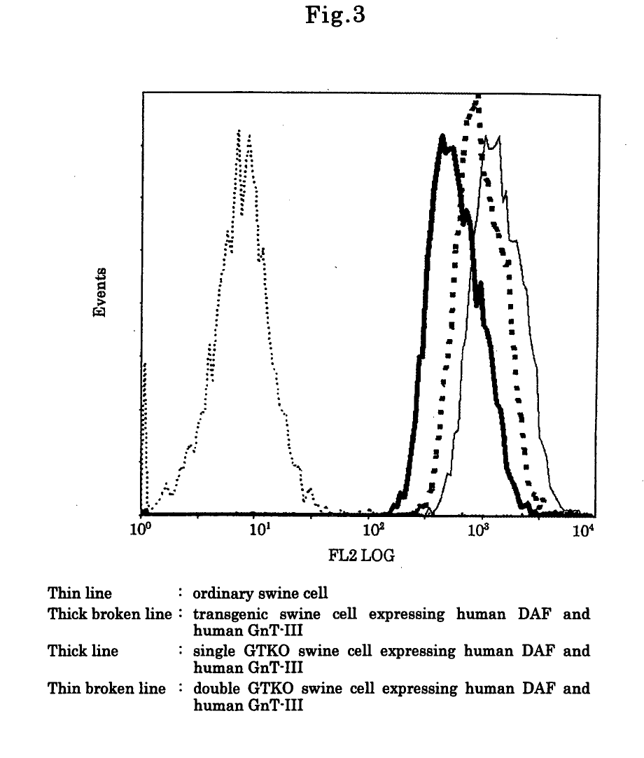

In the GTKO swine cells expressing human DAF and human GnT-III and the GTKO swine cells expressing human DAF, α-Gal antigen was analyzed by FACS by using biotin labeled GSIB4 and phycoerythrin labeled streptoavidin, and expression of α-Gal antigen was not confirmed in the double GTKO swine cells. On the other hand, the α-Gal antigen amount of the single GTKO swine cells was decreased as compared with the level of ordinary swine, but it was confirmed that a large quantity was remaining (FIG. 3).

### Example 7

### Complement Reaction Resistance of Double GTKO Swine Cells Expressing Human Complement Inhibitor and/or Human GnT-III

Resistance test to human complement was carried out in cells sampled from ordinary swine (wild type), transgenic swine expressing human DAF, transgenic swine expressing human DAF and human GnT-III, the double GTKO swine expressing human DAF, and the double GTKO swine expressing human DAF and human GnT-III. Results are shown in Table 1.

Cell mortality of ordinary swine was 100%, but cell mortality was decreased in transgenic swine expressing human DAF, transgenic swine expressing human DAF and human GnT-III, the double GTKO swine expressing human DAF, and the double GTKO swine expressing human DAF and human GnT-III. In particular, cell mortality was decreased more in the double GTKO swine expressing human DAF, and cell mortality was further decreased in the double GTKO swine expressing human DAF and human GnT-III.

It has been hence confirmed that cells of the double GTKO swine expressing human DAF do not react with anti-α-Gal antigen antibody in human serum, and/or suppress complement reaction induced by reaction of antigen other than α-Gal antigen and antibody in human serum.

Further, it is found that cells of the double GTKO swine expressing human DAF and human GnT-III do not react with anti-α-Gal antigen in human serum, do not cause reaction with antigen other than α-Gal antigen of swine cells, and suppress complement reaction induced by reaction of antibody in human serum and xenoantigen of swine cell.

It has been hence confirmed that cells of the double GTKO swine expressing human DAF suppress reaction with human serum inducing the acute rejection characteristic to xenotransplantation. It has been also confirmed that cells of the double GTKO swine expressing human DAF and human GnT-III more efficiently suppress reaction with human serum inducing the acute rejection characteristic to xenotransplantation.

TABLE 1
Complement dependent cell mortality of swine cells (%)

Sample human serum
concentration

| Sample cell | 20% | 40% |
|---|---|---|
| Ordinary swine | 100 | 100 |
| Transgenic swine expressing human | 22 | 39 |
| DAF | | |
| Transgenic swine expressing human | 16 | 28 |
| DAF and human GnT-III | | |
| Double GTKO swine expressing | 3 | 10 |
| human DAF | | |
| Double GTKO swine expressing | <1 | <1 |
| human DAF and human GnT-III | | |

The experiments were conducted in the following methods.

### Identification of Transgene DNA

DNA of human DAF, and DNA of human GnT-III and GTKO targeting vector incorporated in the swine cells and swine genomes of the invention were identified in the following conditions.
(1) Identification of DNA of Human DAF

Using DNA extracted from the sample swine as template, PCR reaction was conducted by using 5′-GGCCTTCCCCCAGATGTACCTAATGCC (SEQ ID NO: 1) derived from human DAF cDNA as sense primer, and its 5′-TCCATAATGGTCACGTTCCCCTTG (SEQ ID NO: 2) as antisense primer (condition; at 94° C. for 30 seconds, at 68° C. for 2 minutes and 30 seconds, 30 times). Each sample was electrophoresed in mini gel (containing ethidium bromide), and presence or absence of the introduced gene was detected and identified.
(2) Identification of DNA of Human GnT-III

Using DNA extracted from the sample swine as template, PCR reaction was conducted by using 5′-GGTGGACGCCTTTGTGGTGTGC (SEQ ID NO: 3) derived from human GnT-III cCDNA as sense primer, and its 5′-GCCGGTGCGGTTCTCATACTGT (SEQ ID NO: 4) as antisense primer, it was identified same as above.
(3) Identification of DNA of GTKO Targeting Vector
   (a) Preparation of Probe for Southern Blotting

In DNA extracted from swine, using CCCTGCTGCCACCTGCTCTAACTCT (SEQ ID NO: 5) and ACTGGGTCTCCCATTGCCATCCTCT (SEQ ID NO: 6) as primers, PCR was conducted (at 94° C. for 30 seconds, at 68° C. for 30 seconds, at 72° C. for 1 minute, 35 cycles: PCR DIG Probe Synthesis Kit, Roche Co.), Dig labeled probe for Southern blotting composed of DNA sequence of 3′ side of swine GT exon 9 was prepared.

(b) Identification of GTKO Targeting Vector by Southern Blotting

Five microgram of genome extracted from the tail of sample swine was digested by EcoRV, and electrophoresed in 1% agarose, and transferred on a membrane. The membrane was hybridized with Dig (digoxigenin) labeled probe obtained by above (a). After washing, alkaline phosphatase labeled anti-Dig antibody and chemical luminescent agent were applied on the membrane to act. In the case of ordinary swine, single GTKO swine and double GTKO swine, respectively, band of 3000 bp only, 3000 bp and 5350 bp, and 5350 bp only were detected.

### Measuring Method of Introduced Gene Product

(1) Measurement of Human DAF Protein

Fibroblast sampled from swine ear was cultivated in 10% bovine fetal serum added DMEM culture medium, and washed and treated in biotin labeled anti-DAF antibody (IA10) and phycoerythrin labeled streptoavidin, and analyzed by FACS (Mol. Reprod. Dev. 2002:61; 302-11).
(2) Measurement of Enzyme Activity of Human GnT-III

Lyzate was prepared by lysis of fibroblast cell of sample swine, and the enzyme activity of human GnT-III was measured in substrate of pyridyl-aminated biantennary-sugar chain (J. Biol. Chem. 2001: 276; 39310-19).
(3) Measurement of α-Gal Antigen

Fibroblast cell of sample swine was treated in biotin labeled GS1B4 and phycoerythrin labeled streptoavidin, and analyzed by FACS.
(4) Resistance Test to Human Complement

Samples taken from ordinary swine (wild type), transgenic swine expressing human DAF, transgenic swine expressing human DAF and human GnT-III, the double GTKO swine expressing human DAF, and the double GTKO swine expressing human DAF and human GnT-III were seeded on 96-hole plate by 1×10⁴ cells each. After cultivation for 24 hours, the culture supernatant was removed, and cultivated in culture medium containing human serum by 20% or 40% (37° C., 1.5 to 3 hours). After cultivation, using the amount of lactate dehydrogenase (LDH) contained in the culture supernatant as index, the number of dead cells was measured (MTX LDH Kit, Kyokuto Seiyaku), and the cell mortality was calculated.

1. A swine cell not expressing α-1,3-galactosyl transferase (GT) but expressing human complement inhibitor and/or human N-acetyl glucosaminyl transferase III (GnT-III).

2. A selecting method of a double GTKO swine cell expressing human complement inhibitor and/or human GnT-III from a mixture of swine cell expressing human complement inhibitor and/or human GnT-III and knocking out one allele of GT gene (single GTKO) and swine cell expressing human complement inhibitor and/or human GnT-III and knocking out both alleles of GT gene (double GTKO) characterized by removing the single GTKO swine cell expressing human complement inhibitor and/or human GnT-III and obtaining the double GTKO swine cell expressing human complement inhibitor and/or human GnT-III by causing
(a) a substance specifically bonding with galactose α-1,3-galactose (α-Gal antigen) bonded with a carrier; or
(b) one substance of a pair of substances having bonding property bonded with a substance specifically bonding with α-Gal antigen, and other substance of the pair of substances having bonding property bonded with a carrier; to act on the mixture.

3. The selecting method of claim 2, wherein the substance specifically bonding with α-Gal antigen is α-Gal antigen specific lectin (Griffonia simplicifolia 1 isolectin B4; GS1 B4) or anti-α antigen antibody.

4. The selecting method of claim 2, wherein the pair of substances having bonding property are combination of avidin (or streptoavidin) and biotin (or iminobiotin, dithiobitin) or combination of concanavalin A and sugars.

5. The selecting method of claim 2, 3 or 4, wherein the carrier is magnetic beads.

6. A selecting method of the double GTKO swine cell expressing human complement inhibitor and/or human GnT-III from a mixture of the single GTKO swine cell expressing human complement inhibitor and/or human GnT-III and the double GTKO swine cell of the same characterized by removing the single GTKO swine cell expressing human complement inhibitor and/or human GnT-III and obtaining the double GTKO swine cell expressing human complement inhibitor and/or human GnT-III by causing anti-human complement inhibitor antibody to act on the mixture, and then anti-α-Gal antibody and complement to act on the resultant mixture.

7. A swine for xenotransplantation having a swine cell not expressing GT and expressing human complement inhibitor and/or human GnT-III.


SEQUENCE LISTING (6 sequences)

SEQ ID NO: 1 - DNA, 27 bp, Artificial Sequence
  Primer derived from human DAF cDNA
ggccttcccccagatgtacctaatgcc

SEQ ID NO: 2 - DNA, 24 bp, Artificial Sequence
  Primer derived from human DAF cDNA
tccataatggtcacgttccccttg

SEQ ID NO: 3 - DNA, 22 bp, Artificial Sequence
  Primer derived from human GnT-III cDNA
ggtggacgcctttgtggtgtgc

SEQ ID NO: 4 - DNA, 22 bp, Artificial Sequence
  Primer derived from human GnT-III cDNA
gccggtgcggttctcatactgt

SEQ ID NO: 5 - DNA, 25 bp, Artificial Sequence
  Synthetic primer
ccctgctgccacctgctctaactct

SEQ ID NO: 6 - DNA, 25 bp, Artificial Sequence
  Synthetic primer
actgggtctcccattgccatcctct